(19) United States
(12) Patent Application Publication
(10) Pub. No.: US 2008/0051726 A1
(43) Pub. Date: Feb. 28, 2008

(54) NEEDLE ASSEMBLY

(75) Inventors: Chien-Yuan LEE, Tamsui Town (TW); Chih-Cheng KAO, Taipei (TW)

Correspondence Address: PATENTTM.US, P. O. BOX 82788, PORTLAND, OR, 97282-0788, US

(73) Assignee: LINFAIR ENGINEERING (HK) CO., LTD., Chai Wan, Hong Kong (CN)

(21) Appl. No.: 11/462,245

(22) Filed: Aug. 3, 2006

(51) Int. Cl. A61M 5/178; A61M 5/32

(52) U.S. Cl. 604/198; 604/164.01

(57) ABSTRACT

An needle assembly includes a barrel having a front opening, a rear opening in communication with the front opening, a front blockage formed and an annular rear stop formed on an inner periphery of the barrel to be adjacent to the rear opening. A needle hub is movably received inside the barrel and having a stop ring securely mounted around the needle hub. The needle hub is alternately positioned by the front blockage and the rear stop. A needle is securely mounted at a distal end of the needle hub to be extended out of the barrel when the stop ring is positioned by the front blockage and to be received inside the barrel when the stop ring is positioned by the rear stop. A tube is connected to the needle hub to be in communication with the needle hub.

### BACKGROUND OF THE INVENTION

1. Field of the Invention

The present invention relates to a needle assembly, and more particularly to a needle assembly having a retractable needle so as to secure safety of the user after the application of the needle assembly.

2. Description of the Prior Art

with reference to FIG. 15, a conventional needle assembly is shown and includes a needle hub (41), a needle (42) securely mounted at a front end of the needle hub (41), a tube (43) securely mounted at a rear end of the needle hub (41) and a barrel (45) provided to allow extension of the needle hub (41) as well as the needle (42). The needle hub (41) has an annular front recess (411) defined in the front end of the needle hub (41) and an annular rear recess (412) defined in the rear end of the needle hub (41). The barrel (45) has an annular protrusion (451) formed on an inner periphery of the barrel (45) to alternately correspond to the front recess (411) and the rear recess (412). Also, a pair of wings (452) is provided on opposite sides of the barrel (45) to facilitate holding of the barrel (45) especially after the needle hub (41) as well as the needle (42) is assembled with the barrel (45).

When the needle hub (41) together with the needle (42) is assembled with the barrel (45), the needle hub (41) as well as the needle (42) is extended into the barrel (45) and the needle (42) is extending out of the barrel (45) with the annular protrusion (451) received in the rear recess (412). While the needle hub (41) is extending into the barrel (45), the needle hub (41) is moved forward to pass through the annular protrusion (451). Then the annular protrusion (451) is received in the rear recess (412) to position the needle hub (41) inside the barrel (45).

After application, the operator is able to pull the needle hub (41) backward to allow the annular protrusion (451) to be received in the front recess (411) of the needle hub (41). Meanwhile, the needle (42) is received inside the barrel (45), which prevents the operator from being injured by the exposed needle (42).

The positioning effect from the barrel (45) to the needle hub (41) is weak so that often the needle hub (41) as well as the needle (42) is still movable inside the barrel (45). That is, the annular protrusion (451) received in rear recess (412) can not provide solid positioning effect to the needle hub (41). Also, the needle hub (41) as well as the needle (42) may be easily pulled out of the barrel (45) due to weak engagement between the annular protrusion (451) and the rear recess (412).

To overcome the shortcomings, the present invention tends to provide an improved needle assembly to mitigate the aforementioned problems.

### SUMMARY OF THE INVENTION

The primary objective of the present invention is to provide an improved needle assembly having therein a stop ring securely mounted on an outer periphery of the needle hub, an annular front stop formed on an inner periphery of the barrel to be adjacent to the front opening so as to define therebetween a first receiving space and an annular rear stop formed on the inner periphery of the barrel to be adjacent to the rear opening so as to define therebetween a second receiving space. Both the first receiving space and the second receiving space are configured to have a dimension corresponding to a dimension of the stop ring so that when the needle hub together with the needle is movable inside the barrel, the first receiving space and the second receiving space provide secured positioning effect to the needle hub, especially when the needle is received inside the barrel.

Another objective of the present invention is that the barrel is sectioned so that the assembly of the needle hub, the stop ring and the needle may be first finished and then the sectioned barrel is assembled to the needle hub, the needle as well as the barrel.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the needle assembly of the present invention;

FIG. 2 is a cross sectional view showing the assembled needle assembly in FIG. 1;

FIG. 3 is an operationally cross sectional view showing the needle is received in the barrel of the assembly of the present invention;

FIG. 4 is a cross sectional view showing that the rear stop of the barrel is tapered to facilitate extension of the needle hub into the barrel;

FIG. 4A is a schematically cross sectional view showing alternate embodiment of the connection device;

FIG. 5 is a cross sectional view showing that the needle hub is conical to facilitate extension of the needle hub into the barrel;

FIG. 6 is a cross sectional view showing that the pair of wings of the barrel is removed;

FIG. 7 is an exploded perspective view showing that the stop ring is C-shaped;

FIG. 8 is a cross sectional view showing that the tube has a sleeve formed on an outer periphery of the tube;

FIG. 9 is a schematically cross sectional view showing that the side face of the confinement ring is tapered;

FIG. 10 is an exploded perspective view of a different embodiment of the needle assembly of the present invention;

FIG. 11 is a cross sectional view showing that the embodiment in FIG. 10 is assembled;

FIG. 12 is a cross sectional view showing that the barrel is sectioned;

FIG. 12A is a schematically cross sectional view showing alternate embodiment of the connection device;

FIG. 13 is a cross sectional view showing that a front stop is provided inside the barrel to enhance the stopping effect of the resilient spring inside the barrel;

FIG. 14 is a cross sectional view showing that the barrel is now provided with a pushbutton mounted around an outer periphery of the barrel; and

FIG. 15 is a schematic cross sectional view of a conventional needle assembly.

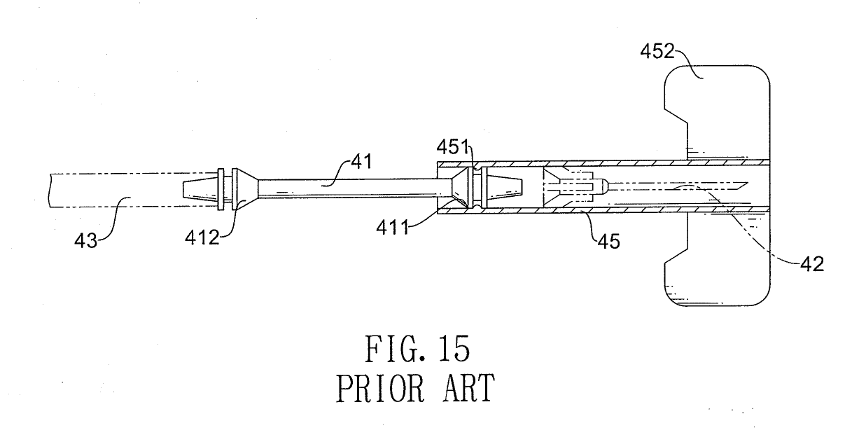

### DETAILED DESCRIPTION OF THE PREFERRED EMBODIMENT

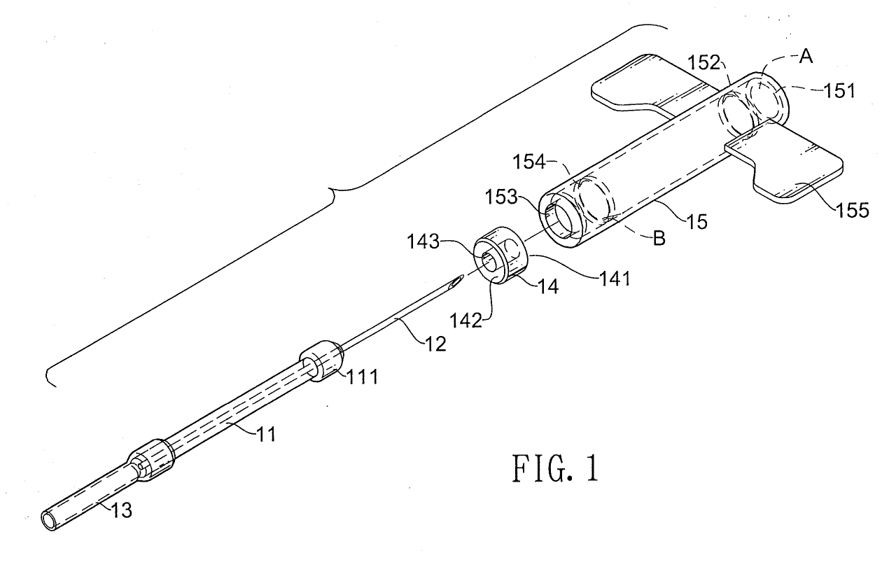

With reference to FIG. 1, it is noted that the needle assembly in accordance with the present invention includes a needle hub (11) having a needle (12) firmly connected to a distal end of the needle hub (11) and a tube (13) securely connected to and communicated with a proximal end of the needle hub (11), a stop ring (14) and a barrel (15).

The needle hub (11) further has a front block (111) formed at a joint between the needle hub (11) and the needle (12). The stop ring (14) has a front side (141) and a rear side (142) with a through hole (143) defined through the front side (141) and the rear side (142). The barrel (15) has a front opening (151), a rear opening (153) communicating with the front opening (151), an annular front stop (152) formed on an inner periphery of the barrel (15) to be close to the front opening (151) so as to define a first receiving space (A) between the front opening (151) and the front stop (152) and an annular rear stop (154) formed on the inner periphery of the barrel (15) to be close to the rear opening (153) so as to define a second receiving space (B) between the rear opening (153) and the rear stop (154). Two wings (155) are respectively formed on two opposite sides of the outer periphery of the barrel (15) to facilitate holding of the entire assembly, especially after being assembled.

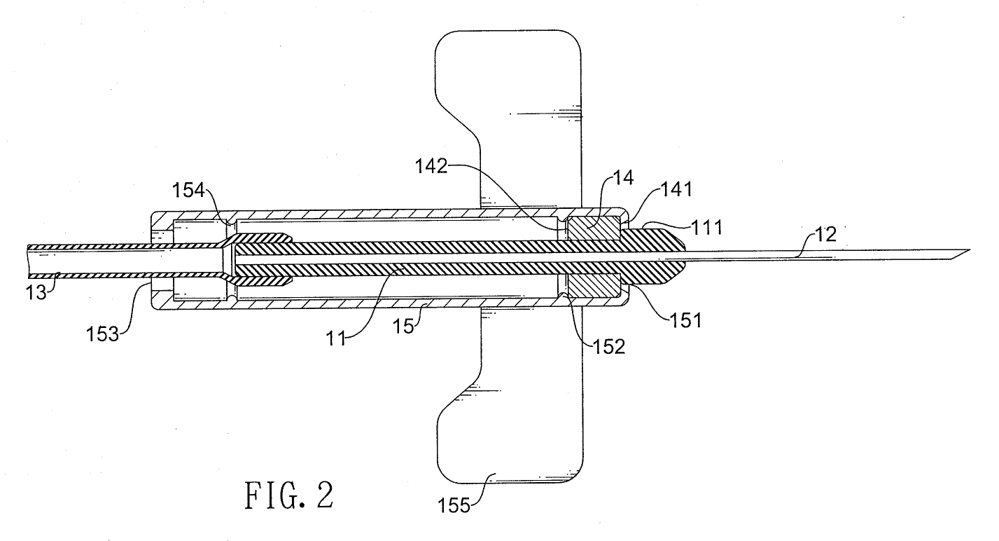

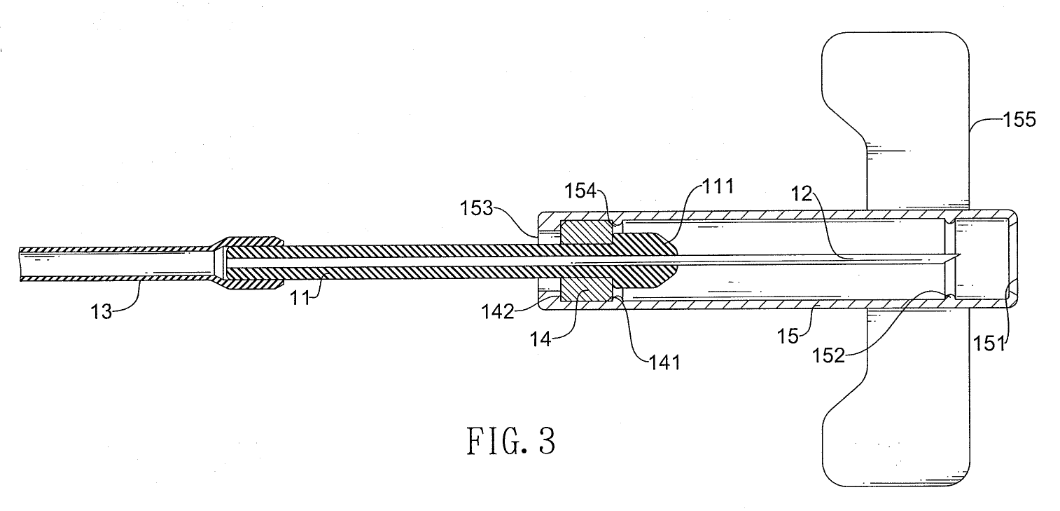

With reference to FIGS. 2 and 3, when the assembly of the present invention is assembled, the stop ring (14) is first firmly mounted around the needle hub (11) to be adjacent to the front block (111) and then the needle hub (11) together with the stop ring (14) is extended into the barrel (15). Due to the resilience of the material which is used to make the barrel (15), the stop ring (14) is able to be forced into the barrel (15) and passed over the rear stop (154). Eventually the stop ring (14) is received in the first receiving space (A) and the needle (12) is extended out of the barrel (15) to allow the entire assembly to be ready for application. Therefore, the tube (13) is able to connect to a container for liquid transmission out of the needle (12).

After use, the operator may pull, by holding the two wings (155), the needle hub (11) backward to retract the needle (12) back into the barrel (15). After the stop ring (14) is received in the second receiving space (B), the needle (12) is safely stored inside the barrel (15) and the operator's safety is secured. That is, because the stop ring (14) is confined by a periphery defining the rear opening (153) and the rear stop (154), the needle (12) is situated inside the barrel (15). As such the operator will no longer be afraid of being injured by the extended needle (12).

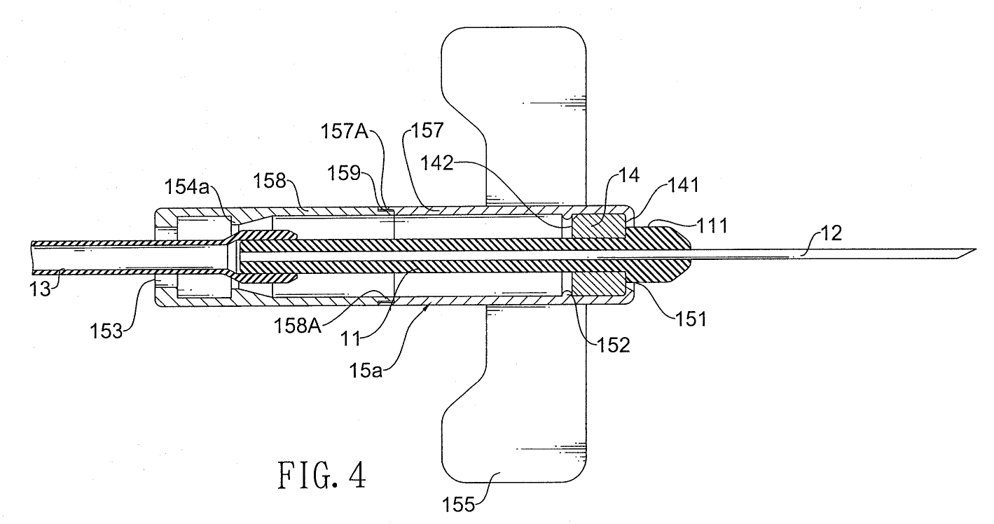

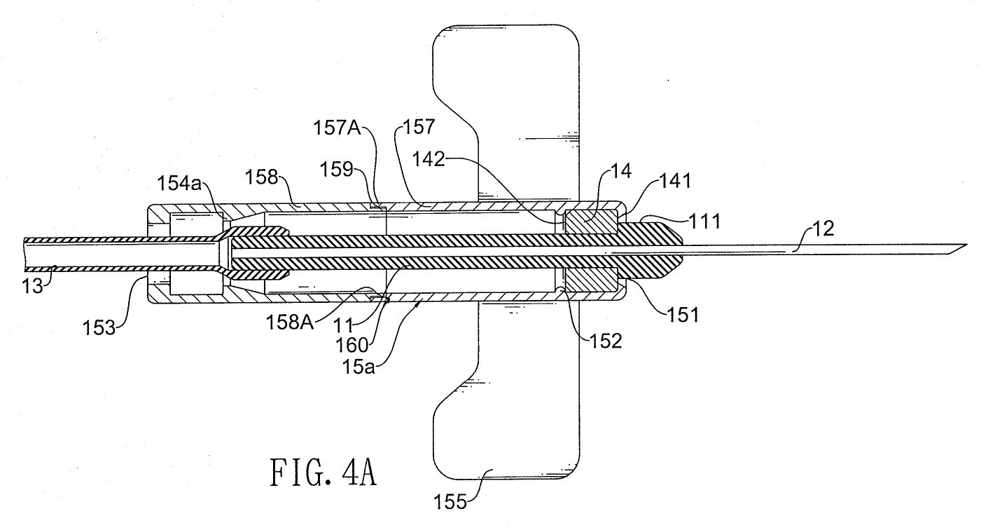

With reference to FIG. 4, in order to facilitate the assembly of the present invention, the barrel (15) may be sectioned. That is the barrel (15) is composed of a front half (157) and a rear half (158). A connection device (159) is provided between the front half (157) and the rear half (158) to securely combine the front half (157) and the rear half (158). Preferably the connection device (159) includes an L shaped extension (157A) formed on a peripheral edge of the front half (157) and an L shaped recess (158A) defined in a peripheral edge of the rear half (158) to correspond to and receive therein the L shaped extension (157A). After the L shaped extension (157A) is received in the corresponding L shaped recess (158A), ultrasonic welding is employed to secure the combination between the front half (157) and the rear half (158). Alternate embodiment of the connection device is that a threading (160) respectively formed on an inner peripheral edge of the front half (157) and an outer peripheral edge of the rear half (158), as shown in FIG. 4A, is employed to secure the connection between the front half (157) and the rear half (158). Furthermore, to facilitate the extension of the needle hub (11) as well as the needle (12) into the barrel (15), the rear stop (154a) is tapered. Furthermore, the needle hub (11) as well as the needle (12) is securely confined inside the barrel (15).

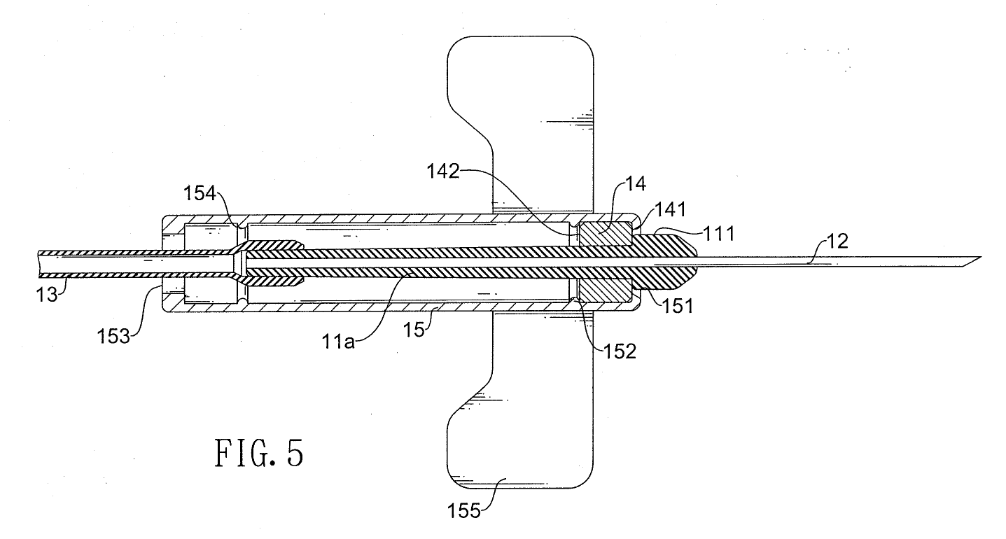

The embodiment shown in FIG. 5 has one big difference than the other embodiments. That is, the needle hub (11a) is conical in dimension so that the needle hub (11a) is easily extended into the barrel (15). That is, the dimension of the needle hub (11a) is reduced toward the rear of the needle hub (11a). In addition, the positioning effect provided to the stop ring (14) by the needle hub (11a) is becoming firmer and firmer as the stop ring (14) advances on the needle hub (11a) toward the front block (111).

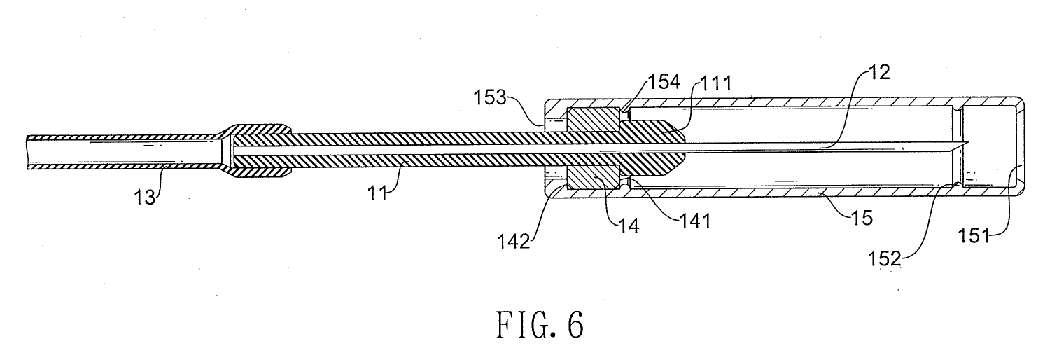

With reference to FIG. 6, as stated earlier that the operator may hold the two wings (155) to apply the needle assembly of the present invention. However, even if the wings (155) are removed, the operator may still directly hold the barrel (15) to apply the needle assembly.

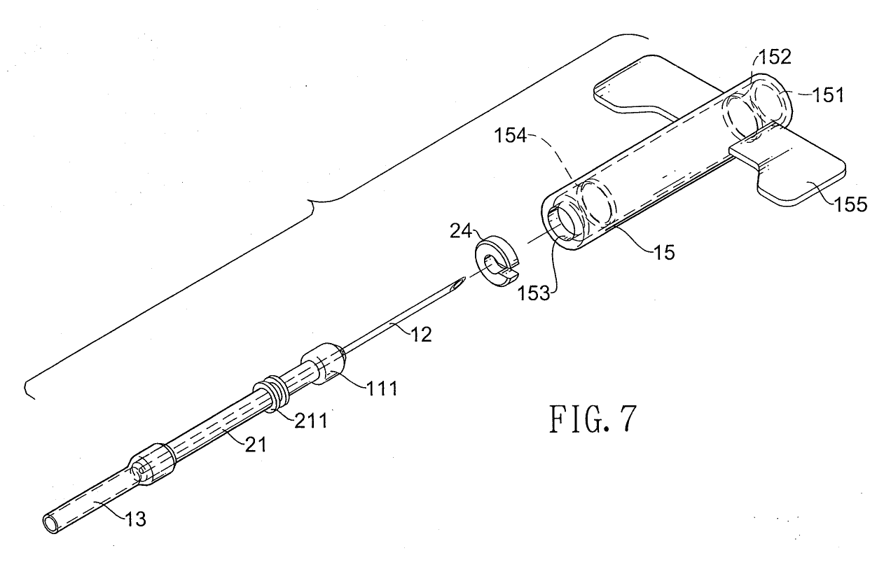

With reference to FIG. 7, most of the structure of the elements are substantially the same as those disclosed in the previous embodiments. The only difference is that the needle hub (21) of this embodiment further has two confinement rings (211) formed on the outer periphery of the needle hub (21). A distance between the two confinement rings (211) corresponds to a radius thickness of the stop ring (24) so that the stop ring (24) is able to be situated between the two confinement rings (211). Furthermore, to accelerate the mounting of the stop ring (24) onto the needle hub (21), the stop ring (24) is C shaped.

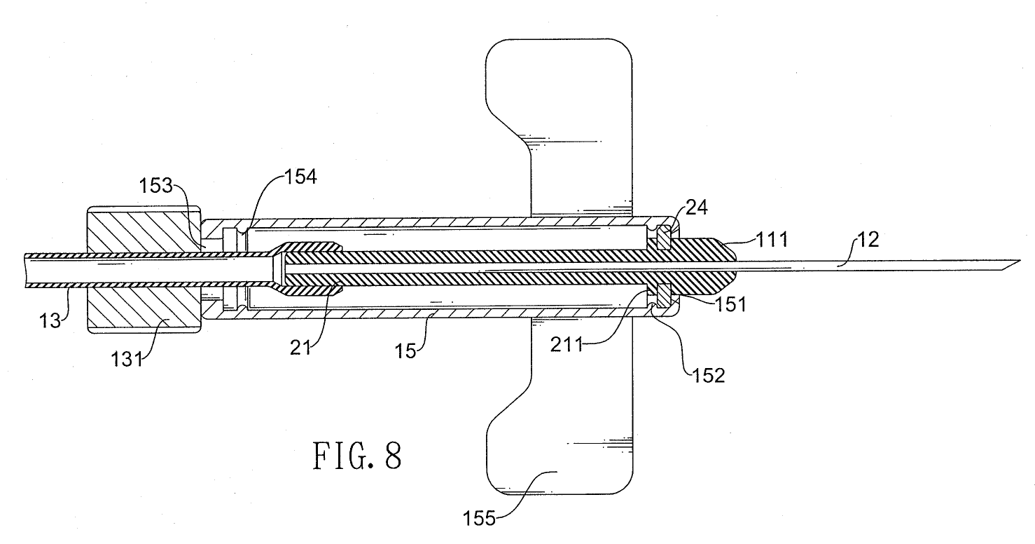

With reference to FIG. 8, it is noted that the needle hub (21) now has only one confinement ring (211) formed on the needle hub (21). A distance between the single confinement ring (211) and the front block (111) corresponds to the radius thickness of the stop ring (24) such that the stop ring (24) may be situated in a space defined between the front block and the confinement ring (211). Still further, the tube (13) may be provided with a sleeve (131) formed on an outer periphery of the tube (13) to facilitate the operator to hold the sleeve (131) to retract the needle hub (21) and the needle (12) into the barrel (15).

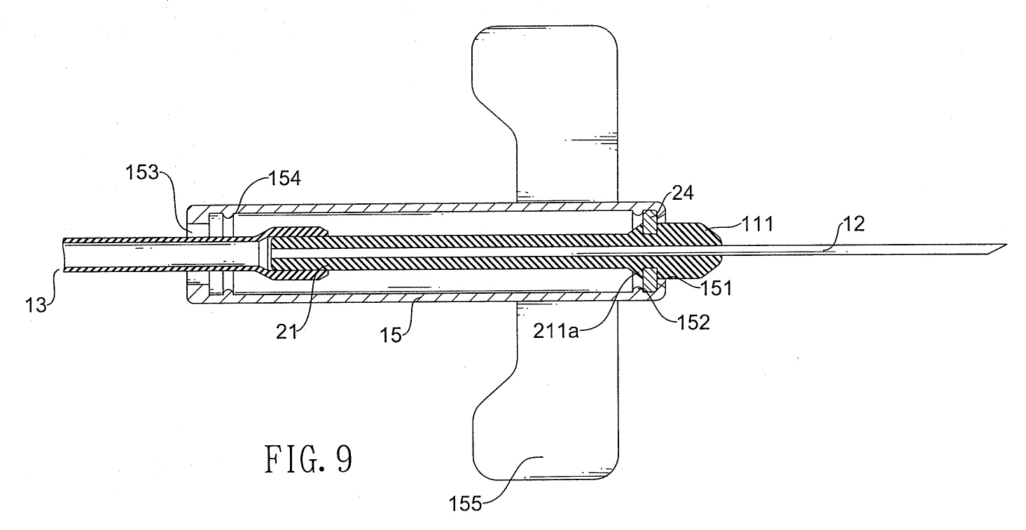

With reference to FIG. 9, in order to save material and to facilitate the extension of the needle hub (21) into the barrel (15), a side face of the confinement ring (211a) is tapered.

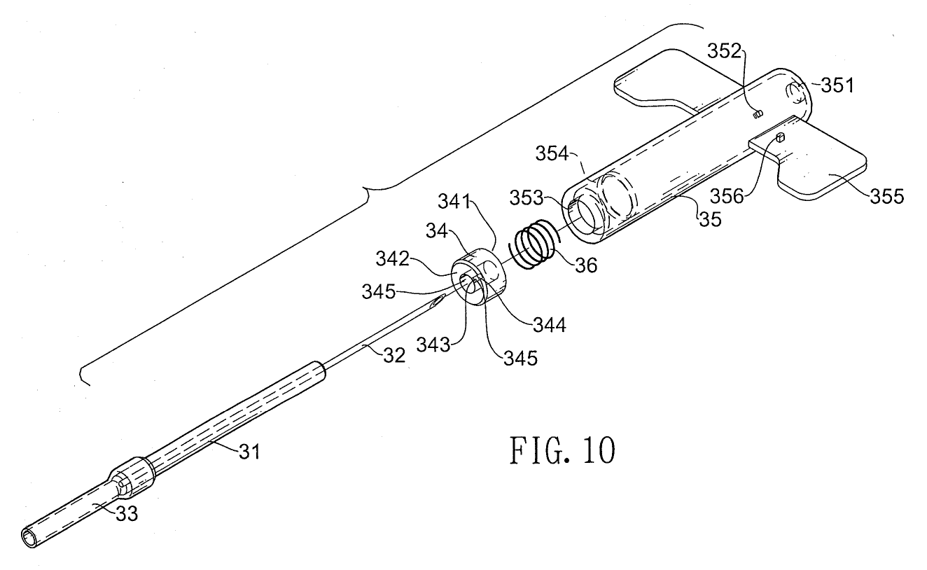

With reference to FIG. 10, in this embodiment, it is noted that the needle hub (31) has a needle (32) firmly mounted at the distal end thereof and a tube (33) securely connected to the proximal end of the needle hub (31). The stop ring (34) has a front side (341), a rear side (342), a through hole (343) defined through the front side (341) and the rear side (342) and an integrally formed snap plate (344) having an L shaped head (345) formed on a free end of the snap plate (344). A spring (36) is provided between the stop ring (34) and a front opening (351) of the barrel (35).

The barrel (35) now has the front opening (351), a rear opening (353) in communication with the front opening (351), a positioning hole (352) defined through the periphery of the barrel (35) to correspond to the L shaped head (345) of the stop ring (34), a rear stop (354) formed adjacent to the rear opening (353), a pair of wings (355) respectively mounted on two opposed sides of the outer periphery of the barrel (15) and a boss (356) formed on a corresponding one of the two wings (355) to correspond to the L shaped head (345) of the stop ring (34).

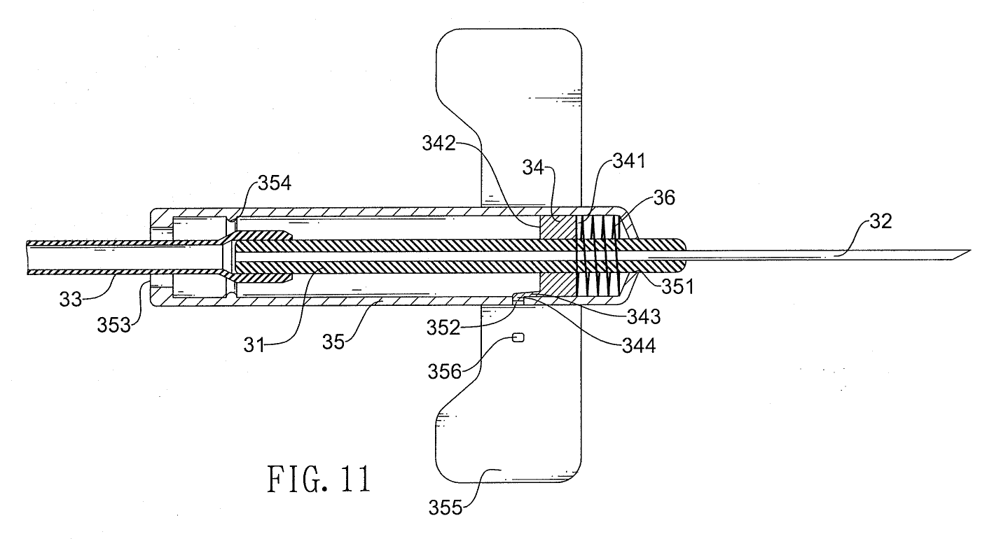

With reference to FIG. 11, when the needle assembly of the present invention is assembled, the stop ring (34) is mounted around the needle hub (31) and the spring (36) is extended into the barrel (35) to be stopped by the front opening (351) of the barrel (35). Thereafter, the needle hub (31) together with the stop ring (34) is forced into the barrel (35) to allow the L shaped head (345) to be in the positioning hole (352). Simultaneously, the spring (36) is compressed between the front opening (351) of the barrel (35) and the stop ring (34). Due to the L shaped head (345) being received in the positioning hole (352), the spring (36) is maintained compressed. Thus the operator is able to hold the two wings (355) to apply the needle assembly of the present invention.

After application, the operator is able to bend the wing (355) to allow the boss (356) to urge against the L shaped head (345) which is received in the positioning hole (352) and eventually be extended into the positioning hole (352). After the L shaped head (345) is pushed into the barrel (35), the resilience force stored in the spring (36) is released and the stop ring (34) is pushed backward. Then the operator is able to force the stop ring (34) to be received in the rear receiving space defined between the rear stop (354) and the rear opening (353). The needle (32) is safely received inside the barrel (35).

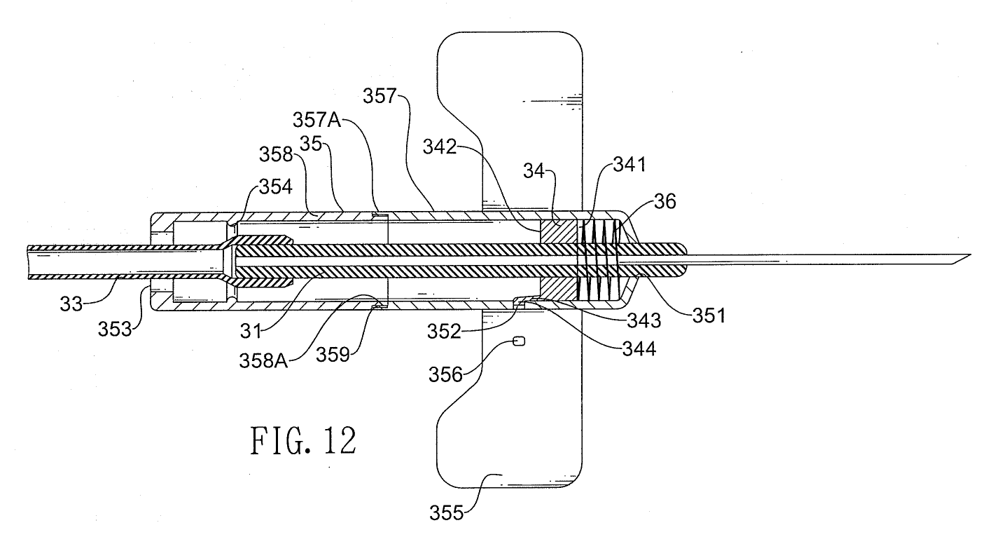

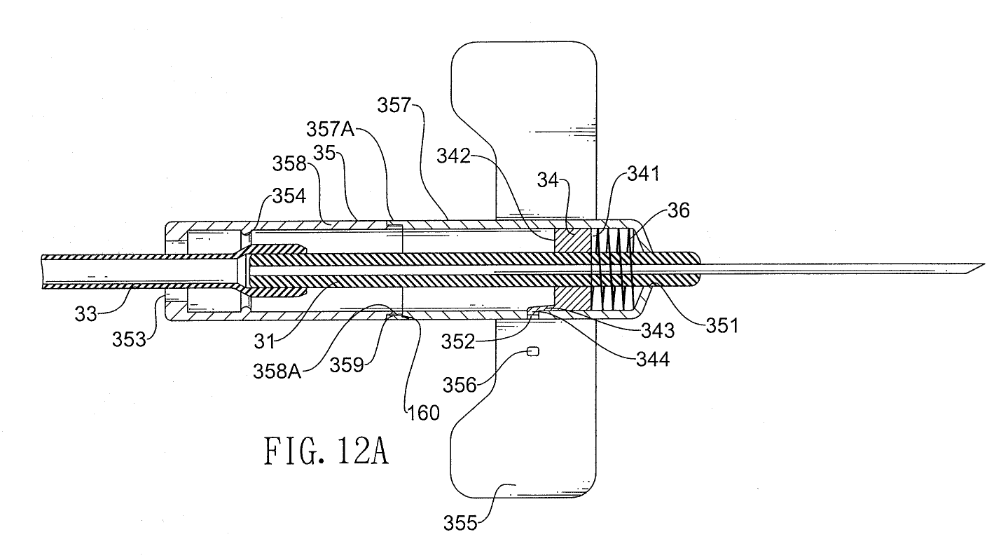

With reference to FIG. 12, in order to facilitate the assembly of the present invention, the barrel (35) may be sectioned. That is the barrel (35) is composed of a front half (357) and a rear half (358). A connection device (359) is provided between the front half (357) and the rear half (358) to securely combine the front half (357) and the rear half (358). Preferably the connection device (359) includes an L shaped extension (357A) formed on a peripheral edge of the front half (357) and an L shaped recess (358A) defined in a peripheral edge of the rear half (358) to correspond to and receive therein the L shaped extension (357A). After the L shaped extension (357A) is received in the corresponding L shaped recess (358A), ultrasonic welding is employed to secure the combination between the front half (357) and the rear half (358). Alternate embodiment of the connection device is that a threading (160) respectively formed on an inner peripheral edge of the front half (357) and an outer peripheral edge of the rear half (358), as shown in FIG. 12A, is employed to secure the connection between the front half (357) and the rear half (358).

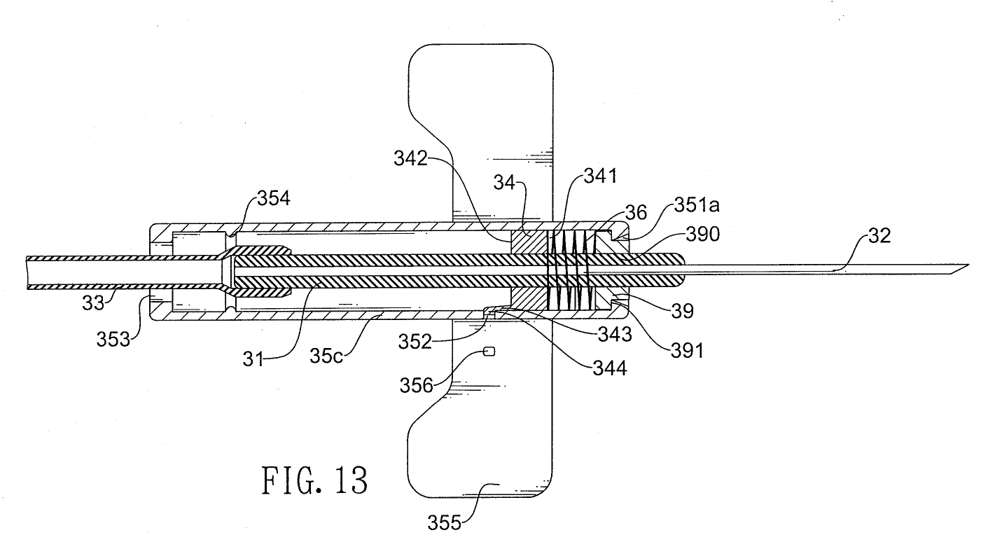

With reference to FIG. 13, in order to enhance the compression to the spring (36), the barrel (35) is provided with an abutting block (39) securely received inside the barrel (35) and abutted against the front opening (351a). The abutting block (39) has a hole (390) defined to allow extension of the needle (32) or the needle hub (31) and a step (391) such that a reduced head of the abutting block (39) is received in the front opening (351a) and an enlarged bottom is received inside the barrel (35) to be engaged with the spring (36).

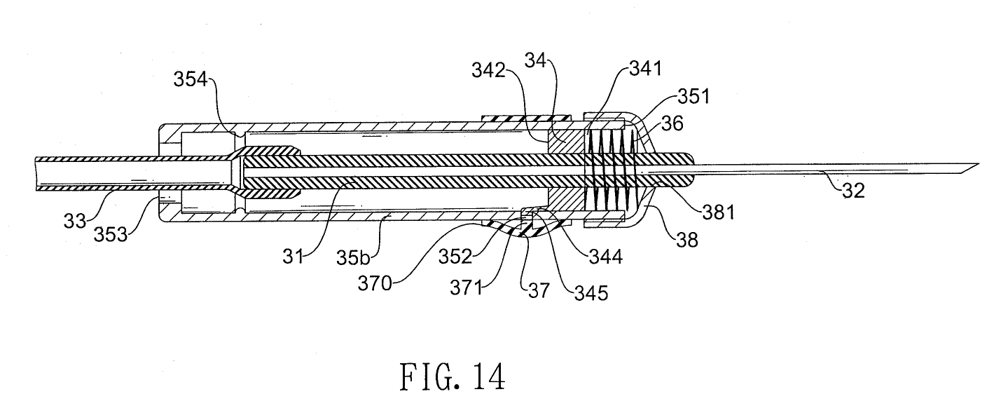

With reference to FIG. 14, it is noted that the boss (356) in FIG. 10 is now changed to a pushbutton (37) mounted around the barrel (35b) and having an annular urging plate (370) extending on the outer periphery of the barrel (35b) and a rod (371) integrally extending from an inner face of the annular urging plate (370) and into the positioning hole (352) of the barrel (35) to abut the L shaped head (345) of the snap plate (344). Furthermore, a cap (38) is provided to the barrel (35) to close the front opening (351). The cap (38) has an extension hole (381) defined to allow the extension of the needle hub (31) or needle (32). After the application of the present invention, the operator is able to press the pushbutton (37) to force the rod (371) to push the L shaped head (345) to leave the positioning hole (352). Thereafter, the spring (36) pushes the stop ring (34) backward inside the barrel (35) to be received in the rear receiving space defined by the rear opening (353) of the barrel (35) and the rear stop (354). Then the needle (32) is safely received inside the barrel (35). Therefore, the operator needs not be afraid of being injured by the protruded needle while handling the needle assembly.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An needle assembly comprising:
a barrel having a front opening, a rear opening in communication with the front opening, a front blockage formed inside the barrel to be adjacent to the front opening and an annular rear stop formed on an inner periphery of the barrel to be adjacent to the rear opening;
a needle hub movably received inside the barrel and having a stop ring securely mounted around the needle hub, the needle hub being alternately positioned by the front blockage and the rear stop, a needle securely mounted and extended through the needle hub to be extended out of the barrel when the stop ring is positioned by the front blockage and to be received inside the barrel when the stop ring is positioned by the rear stop; and
a tube connected to the needle hub to be in communication with the needle hub.

2. The needle assembly as claimed in claim 1, a first receiving space is defined between the front opening and the front stop inside the barrel to correspond to and receive therein the stop ring and a second receiving space is defined between the rear opening and the rear stop inside the barrel to correspond to and receive therein the stop ring to finish retraction of the needle.

3. The needle assembly as claimed in claim 1, wherein the front blockage is an annular front stop formed on the inner periphery of the barrel.

4. The needle assembly as claimed in claim 2, wherein the front blockage is an annular front stop formed on the inner periphery of the barrel.

5. The needle assembly as claimed in claim 1, wherein two wings are provided on two opposite sides of an outer periphery of the barrel to facilitate holding of the barrel.

6. The needle assembly as claimed in claim 3, wherein the needle hub has a front block formed between the needle hub and the needle to be abutted against the stop ring and extended out of the barrel.

7. The needle assembly as claimed in claim 4, wherein the needle hub has a front block formed between the needle hub and the needle to be abutted against the stop ring and extended out of the barrel.

8. The needle assembly as claimed in claim 5, wherein the needle hub has a front block formed between the needle hub and the needle to be abutted against the stop ring and extended out of the barrel.

9. The needle assembly as claimed in claim 4, wherein the rear stop is tapered to facilitate movement of the stop ring into the second receiving space and stop the stop ring.

10. The needle assembly as claimed in claim 8, wherein the rear stop is tapered to facilitate movement of the stop ring into the second receiving space and stop the stop ring.

11. The needle assembly as claimed in claim 2, wherein the barrel is sectioned and a connection device is provided to secure assembly of the sectioned barrel.

12. The needle assembly as claimed in claim 9, wherein the barrel is composed of a front half and a rear half, the connection device includes an L shaped extension formed on a peripheral edge of the front half and an L shaped recess defined in a peripheral edge of the rear half to correspond to and receive therein the L shaped extension such that after the L shaped extension is received in the corresponding L shaped recess securing elements are employed to secure connection between the front half and the rear half.

13. The needle assembly as claimed in claim 4, wherein the needle hub is tapered to facilitate movement of the stop ring.

14. The needle assembly as claimed in claim 5, wherein the needle hub is tapered to facilitate movement of the stop ring.

15. The needle assembly as claimed in claim 6, wherein the stop ring is C shaped.

16. The needle assembly as claimed in claim 8, wherein the stop ring is C shaped.

17. The needle assembly as claimed in claim 2, wherein the front blockage is composed of a positioning hole defined through a side of the barrel and a boss formed on a side face of a corresponding one of the two wings to correspond to the positioning hole, a spring is compressibly provided between the front opening and the stop ring which has an integrally formed snap plate having an L shaped head formed on a free end of the snap plate to detachably received in the positioning hole so that when the L shaped head is received in the positioning hole, the spring is compressed and when the L shaped head is away from the positioning hole via the boss, the needle hub is pushed by the spring and move rearward inside the barrel to have the needle received in the barrel.

18. The needle assembly as claimed in claim 17, wherein the barrel is sectioned and a connection device is provided to secure assembly of the sectioned barrel, the barrel is composed of a front half and a rear half, the connection device includes an L shaped extension formed on a peripheral edge of the front half and an L shaped recess defined in a peripheral edge of the rear half to correspond to and receive therein the L shaped extension such that after the L shaped extension is received in the corresponding L shaped recess a securing element is employed to secure connection between the front half and the rear half.

19. The needle assembly as claimed in claim 18, wherein to enhance the compression to the spring, the barrel is provided with an abutting block securely received inside the barrel and abutted against the front opening, the abutting block has a hole defined to allow extension of the needle and a step such that a reduced head of the abutting block is received in the front opening and a bottom of the abutting block is received inside the barrel to be engaged with the spring.

20. The needle assembly as claimed in claim 2, wherein the front blockage is composed of a positioning hole defined through a side of the barrel and a pushbutton formed on an outer periphery of the barrel, a spring is compressibly provided between the front opening and the stop ring which has an integrally formed snap plate having an L shaped head formed on a free end of the snap plate to detachably received in the positioning hole, the pushbutton has an urging plate extending on the outer periphery of the barrel and a rod integrally extending into the positioning hole of the barrel to abut the L shaped head of the snap plate so that when the L shaped head is received in the positioning hole, the spring is compressed and when the L shaped head is away from the positioning hole via the rod due to movement of the pushbutton, the needle hub is pushed by the spring and move rearward inside the barrel to have the needle received in the barrel.
